(19) United States
(12) Patent Application Publication
(10) Pub. No.: US 2006/0147515 A1
(43) Pub. Date: Jul. 6, 2006

(54) Bioactive dispersible formulation

(76) Inventors: Zhongzhou Liu, Shanghai (CN); Yan Song, Shanghai (CN)

Correspondence Address: FISH & RICHARDSON PC, P.O. BOX 1022, MINNEAPOLIS, MN, 55440-1022, US

(21) Appl. No.: 11/292,322

(22) Filed: Dec. 1, 2005

Related U.S. Application Data
(60) Provisional application No. 60/632,703, filed on Dec. 2, 2004.

(51) Int. Cl. A61K 31/12; A61K 9/48; A61K 9/20

(52) U.S. Cl. 424/451; 424/464; 514/690

(57) ABSTRACT

A composition containing an effective amount of a water-insoluble bioactive agent and two surfactants having different HLB values, the difference between the HLB values of the two surfactants being greater than 5. This invention also relates to a method of prepare such a composition.

### CROSS REFERENCE TO RELATED APPLICATIONS

Pursuant to 35 USC § 119(e), this application claims priority to U.S. Provisional Application Serial No. 60/632,703, filed Dec. 2, 2004, the contents of which are incorporated herein by reference.

### BACKGROUND

Aqueous formulations can be advantageous over solid formulations. For example, a patient with sore throat would prefer to drink an aqueous formulation so as to avoid the pain from swallowing a tablet or a capsule. However, it is difficult or even impossible to prepare conventional aqueous formulations for bioactive agents that are insoluble in water (i.e., having a solubility lower than 0.1 mg/ml).

To solve this problem, aqueous dispersions containing small particles of water-insoluble bioactive agents have been prepared. As an example, one can place in water a composition containing a water-insoluble bioactive agent and a surfactant to obtain an aqueous dispersion. Desirably, a composition used to prepare such a dispersion has high dispersing capacity, i.e., it can form a dispersion in a short period of time. It is also desirable that such a dispersion has high suspending capability, i.e., small particles in the dispersion remain evenly sized and evenly distributed over a long period of time.

### SUMMARY

This invention is based on unexpected discoveries that (1) a composition containing irisquinone (a water-insoluble bioactive agent) and two surfactants having different HLB (hydrophilic-lipophilic balance) values exhibits high dispersing capacity, and (2) the dispersion prepared from the composition exhibits high suspending capability.

Thus, one feature of this invention is a composition containing a water-insoluble bioactive agent and at least two surfactants having different HLB values, the difference between the HLB values of the two surfactants being greater than 5 (e.g., 10 or higher). Preferably, one surfactant has an HLB value greater than 10 and the other has an HLB value smaller than or equal to 10. The bioactive agent can be in either a solid or a liquid form. When the bioactive agent is a liquid (i.e., is a liquid at room temperature or becomes a liquid during processing), the composition further contains an absorbent. The total amount of the two surfactants can be 0.01 to 0.3 parts per part of the bioactive agent by weight, and the amount of the absorbent, if present, can be 0.5 to 10.0 parts per part of the bioactive agent by weight.

In one embodiment, the bioactive agent is irisquinone, which can be irisquinone A, irisquinone B, or a mixture thereof (e.g., a mixture containing 80 to 95% by weight irisquinone A).

Another aspect of this invention is a method of preparing a composition, which can be used to make a water-insoluble bioactive agent containing dispersion. The method includes selecting two surfactants based on the difference between their HLB values being greater than 5, and blending the two surfactants and a water-insoluble bioactive agent to obtain a composition. The blending step can be performed under high-speed shearing. The method may further include one or more additional steps, such as freeze-smashing the composition or compressing the composition to form a tablet.

The details of one or more embodiments of the invention are set forth in the description below. Other features, objects, and advantages of the invention will be apparent from the description and from the claims.

### DETAILED DESCRIPTION

This invention relates to a composition containing a water-insoluble bioactive agent and two surfactants having different HLB values.

Examples of the water-insoluble bioactive agent include, but are not limited to, irisquinone, prednisone acetate, ibuprofen, ketoprofen, naproxen, domperidone, indometacin, ranitidine, famotidine, paclitaxel, and hydroxycamptothecin. The term “surfactant” refers to a substance that tends to physically adhere to the surface of a water-insoluble bioactive agent and change its physical properties. Examples of the two surfactants include, but are not limited to, sugar esters, polyoxyethylene sorbitan fatty acid esters, polyoxyethylene fatty acid esters, sorbitan fatty acid esters, lecithin, polyethylene glycol fatty acids esters, polyethylene glycol glycerol fatty acid esters, propylene glycol fatty acid esters, sodium lauryl sulfate, and poloxamer 188. The surfactant may have a hydrophilic or lipophilic property, which is attributed to hydrophilic and lipophilic groups attached to the surfactant. This property can be characterized by its HLB value, which is an expression of the balance of the size and strength of the hydrophilic and lipophilic groups of the surfactant. See, e.g., K. R. Lange, Surfactants: A Practical Handbook, Hanser Gardner Publications, Inc. 1999. Generally, a surfactant having an HLB value in the range 0-10 is predominantly lipophilic, and a surfactant having a HLB value greater than 10 is predominantly hydrophilic. The HLB values of commonly-used surfactants are well known in the art. Listed below are the HLB values of some surfactants.

HLB values of some commonly-used surfactants

| Surfactant | HLB Value |
|---|---|
| Sucrose stearate | 3 |
| Lecithin | 3.5 |
| Glycerol monostearate | 3.8 |
| Span 80 | 4.3 |
| Sucrose monostearate | 12 |
| Tween 80 | 15 |
| Sodium lauryl sulfate | 40 |

To prepare the composition of this invention, one first select two surfactants, the difference of the HLB values of which is greater than 5. Preferably, one of the two surfactants is a hydrophilic surfactant having a LHB vaule greater than 10 (such as Tween 80, sucrose monostearate, or sodium lauryl sulfate); and the other is a lipophilic surfactant having a HLB value smaller than or equal to 10 (such as sucrose stearate, lecithin, Span 80, or glycerol monostearate). Preferred surfactant pairs are Tween 80 and sucrose stearate, sucrose monostearate and lecithin, sodium lauryl sulfate and sucrose stearate, Tween 80 and Span 80, Tween 80 and lecithin, sucrose monostearate and glycerol monostearate, and Tween 80 and glycerol monostearate.

One then blends the two selected surfactants and a bioactive agent, the amount of the surfactants preferably being 0.01 to 0.3 parts per part of the bioactive agent by weight. This step can be performed under high-speed shearing to break the bioactive agent into small particles and evenly mix it with the surfactants. The resulting mixture can be further frozen and smashed into even smaller particles having a diameter of 150 μm.

The bioactive agent can be a liquid or a solid having a low melting point (e.g., irisquinone), which is molten and turns liquid during processing, as the temperature rises due to mechanical friction. In this case, it is advantageous to also include at least one absorbent in the composition. The amount of the absorbent can be 0.5 to 10 parts per part of the bioactive agent by weight, preferably from 2.5 to 3 parts per part of the bioactive agent by weight. An absorbent is a water-insoluble substance (having a solubility lower than 0.1 mg/ml) with a large surface area that can absorb and retain a liquid bioactive agent. Examples of a suitable absorbent include, but are not limited to, magnesium oxide, magnesium carbonate, silicon dioxide, magnesium aluminum silicate, calcium carbonate, calcium phosphate, calcium hydrogen phosphate, calcium oxide, calcium hydrogen carbonate, aluminum hydroxide, magnesium hydroxide, Kaolin, or the mixtures thereof.

It is well known in the art that a dispersion tends to have high suspending capability, when it is made from a composition having a tapped density approximately equal to the density of water. Tapped density is the apparent density of a volume of powder obtained when its receptacle is tapped. It is preferred that the composition of this invention have a tapped density approximately equal to the density of water (e.g., 0.8-0.95 g/cm³). Such a composition can be prepared by adjusting the amounts of the surfactants and the absorbent (if present) relative to that of the bioactive agent.

One or more additives, such as a disintegrator, a diluent, or a dispensing agent, can also be included in the composition. A disintegrator is highly hydrophilic and expandable when contacting water. It facilitates disintegration of the composition and release of the bioactive agent from the composition. Examples of a suitable disintegrator include, but are not limited to, sodium starch glycolate, croscarmellose sodium, polyvinyl pyrrolidone, low-substituted hydroxypropyl cellulose, croscarmellose calcium, and alginate sodium. A diluent is a water-disintegratable, compressible agent and provides desired moldability and integrity. Examples of a suitable diluent include, but are not limited to microcrystalline cellulose, powder cellulose, lactose, starch, mannitol, sucrose, dextrose, sorbitol, maltose, xylitol, and a mixture thereof. A dispersing agent prevents adherence and friction of particles of a bioactive agent. Examples of a suitable dispersing agent include, but are not limited to, silicon oxide, starch, and tale.

It is recognized by a skilled person in the art that an additive can be added at any stage in the process of preparing the composition. For example, one can mix an additive together with a bioactive agent and two surfactants. Alternatively, one can dry-blend an additive with an already-mixed composition containing a bioactive agent and two surfactants.

The composition of the present invention can be in the form of granule, dispersion, capsule, or tablet. For example, it can be an aqueous dispersion, i.e., water containing a water-insoluble bioactive agent and two surfactants. Such a dispersion and other embodiments of this invention can be prepared by methods known in the art. For example, one can compress a mixture containing a water-insoluble bioactive agent and two surfactants to form a tablet.

The composition, in any of the forms described above, can be readily used or be further processed. For example, a patient can readily drink a commercially available dispersion or can prepare a dispersion from a tablet himself before oral administration.

All of the above-mentioned bioactive agents, surfactants, absorbents, disintegrators, diluents, and dispersing agents can be purchased from commercial sources, e.g., Sigma-Aldrich Co, or can be prepared by methods well known in the art.

Without further elaboration, it is believed that the above description has adequately enabled the present invention. The following specific examples are, therefore, to be construed as merely illustrative, and not limitative of the remainder of the disclosure in any way whatsoever. All of the publications cited herein are hereby incorporated by reference in their entirety.

### EXAMPLE 1

Irisquinone (Shangdong Xinhua Pharma. Co. Ltd.), sucrose monostearate, Tween 80, silicon dioxide, and magnesium oxide (amounts indicated in Table 1A below) were mixed under high-speed shearing. The mixture was refrigerated at −4° C., smashed, and sieved through 100 meshes. The particles thus obtained were then dry-blended with microcrystalline cellulose, lactose, starch, and croscarmellose sodium (amounts also indicated in Table 1A below), granulated, and compressed to produce 1000 tablets.

TABLE 1A
Formulation containing two surfactants (sucrose stearate,
HLB = 3; Tween 80, HLB = 15)

| | | |
|---|---|---|
| Irisquinone | 59.7 | g |
| Sucrose stearate | 4.8 | g |
| Tween 80 | 3.0 | g |
| Silicon dioxide | 25.5 | g |
| Magnesium oxide | 20.7 | g |
| Microcrystalline cellulose | 132.0 | g |
| Lactose | 29.5 | g |
| Starch | 20 | g |
| Croscarmellose sodium | 4.8 | g |

For comparison, 1000 tablets containing only one surfactant were prepared using the materials listed in Table 1B in a similar manner.

TABLE 1B
Formulation containing one surfactant (sucrose stearate, HLB = 3)

| | | |
|---|---|---|
| Irisquinone | 59.4 | g |
| Sucrose stearate | 7.8 | g |
| Silicon dioxide | 25.5 | g |
| Magnesium oxide | 20.7 | g |
| Microcrystalline cellulose | 132.0 | g |
| Lactose | 29.5 | g |
| Starch | 20 | g |
| Croscarmellose sodium | 4.8 | g |

The dispersing capacity of a number of tablets and suspending capability of the dispersions prepared from the tablets were determined. Briefly, each tested tablet was placed in 100 ml of water at 15-25° C. The disintegrating and dispersing process was observed and the time needed to form a dispersion that could completely flow through a 710 μm sieve was measured. The shorter time needed to form a dispersion indicated that the mixture had a higher dispersing capacity. 50 ml of the above dispersion was then placed in a graduated flask, shaken for 1 min, and allowed to sit at 15-25° C. for 3 hrs. The sedimentation ratio was calculated as the ratio of the height of the sediment in the flask to the height of the dispersion. The greater the sedimentation ratio, the higher the suspending capability of the dispersion. A sedimentation ratio greater than or equal to 0.90 indicated that a dispersion had high suspending capability.

The dispersing capacity of a number of tablets and the suspending capability of the dispersions prepared from the tablets were determined according to the methods described above.

Unexpectedly, the results show that the tablets containing two surfactants (see Table 1A) were disintegrated faster than those containing only one surfactant (see Table 1B), and that the dispersions prepared from the former tablets had a higher sedimentation ratio than those prepared from the latter tablets. More specifically, the former tablets were disintegrated within 15 seconds to form evenly suspending dispersions each having a sedimentation ratio of 0.94, and the latter tablets disintegrated in water within 20 seconds to form oily dispersions each having a sedimentation ratio of 0.90. Note that the “oily” feature is not desirable.

### EXAMPLE 2

1000 tablets were prepared using the materials listed in Table 2 according to the method described in Example 1.

TABLE 2
Formulation containing two surfactants (sucrose monostearate,
HLB = 12; sucrose distearate, HLB = 3)

| | | |
|---|---|---|
| Irisquinone | 52.5 | g |
| Sucrose monostearte | 8.7 | g |
| Sucrose distearte | 3.0 | g |
| Silicon dioxide | 23.1 | g |
| Magnesium oxide | 29.1 | g |
| Microcrystalline cellulose | 87.3 | g |
| Maltose | 67.3 | g |
| Starch | 20 | g |
| Croscarmellose sodium | 9.0 | g |

The dispersing capacity of a number of tablets and the suspending capability of the dispersions prepared from the tablets were determined according to the methods described in Example 1. The results show that all tested tablets were disintegrated in water within 15 seconds to form oily dispersions each having a sedimentation ratio of 0.93.

### EXAMPLE 3

1000 tablets were prepared using the materials listed in Table 3 according to the method described in Example 1.

TABLE 3
Formulation containing two surfactants (sucrose monostearate,
HLB = 12; glycerol monostearate, HLB = 38)

| | | |
|---|---|---|
| Irisquinone | 52.5 | g |
| Sucrose monostearate | 8.7 | g |
| Glycerol monostearate | 3.0 | g |
| Silicon dioxide | 23.1 | g |
| Magnesium oxide | 29.1 | g |
| Microcrystalline cellulose | 87.3 | g |
| Maltose | 67.3 | g |
| Starch | 20 | g |
| Croscarmellose sodium | 9.0 | g |

The dispersing capacity of a number of tablets and the suspending capability of the dispersions prepared from the tablets were determined according to the methods described in Example 1. The results show that all tested tablets were disintegrated in water within 12 seconds to form evenly suspending dispersions each having a sedimentation ratio of 0.97.

### EXAMPLE 4

1000 tablets were prepared using the materials listed in Table 4 according to the method described in Example 1.

TABLE 4
Formulation containing two surfactants
(lecithin, HLB = 3.8; Tween 80, HLB = 15)

| | | |
|---|---|---|
| Irisquinone | 60.1 | g |
| Lecithin | 1.5 | g |
| Tween 80 | 1.5 | g |
| Silicon dioxide | 30.5 | g |
| Calcium hydrogen phosphate | 36.2 | g |
| Microcrystalline cellulose | 98.3 | g |
| Lactose | 65.9 | g |
| Croscarmellose sodium | 6.0 | g |

The dispersing capacity of a number of tablets and the suspending capability of the dispersions prepared from the tablets were determined according to the methods described in Example 1. The results show that all tested tablets were disintegrated in water within 13 seconds to form evenly suspending dispersions each having a sedimentation ratio of 0.91.

### EXAMPLE 5

1000 tablets were prepared using the materials listed in Table 5 according to the method described in Example 1.

TABLE 5
Formulation containing two surfactants
(lecithin, HLB = 3.5; poloxamer 188, HLB = 29)

| | | |
|---|---|---|
| Irisquinone | 62.8 | g |
| Lecithin | 9.0 | g |
| Poloxamer 188 | 8.2 | g |
| Silicon dioxide | 35.4 | g |
| Calcium carbonate | 30.0 | g |
| Microcrystalline cellulose | 116.5 | g |
| Lactose | 29.6 | g |
| Sodium starch Glycolate | 8.5 | g |

The dispersing capacity of a number of tablets and the suspending capability of the dispersions prepared from the tablets were determined according to the methods described in Example 1. The results show that all tested tablets were disintegrated in water within 14 seconds to form evenly suspending dispersions each having a sedimentation ratio of 0.93.

### EXAMPLE 6

1000 tablets were prepared using the materials listed in Table 6 according to the method described in Example 1.

TABLE 6
Formulation containing two surfactants
(lecithin, HLB = 3.8; Tween 80, HLB = 15)

| | | |
|---|---|---|
| Irisquinone | 30.0 | g |
| Lecithin | 0.15 | g |
| Tween 80 | 0.15 | g |
| Silicon dioxide | 12.3 | g |
| Magnesium carbonate | 12.2 | g |
| Microcrystalline cellulose | 142.9 | g |
| Mannitol | 97.3 | g |
| Croscarmellose sodium | 2.0 | g |
| Sodium starch glycolate | 3.0 | g |

The dispersing capacity of a number of tablets and the suspending capability of the dispersions prepared from the tablets were determined according to the methods described in Example 1. The results show that all tested tablets were disintegrated in water within 13 seconds to form evenly suspending dispersions each having a sedimentation ratio of 0.92.

### EXAMPLE 7

1000 tablets were prepared using the materials listed in Table 7 according to the method described in Example 1.

TABLE 7
Formulation containing two surfactants
(sucrose stearate, HLB = 3; poloxamer 188, HLB = 29)

| | | |
|---|---|---|
| Irisquinone | 53.8 | g |
| Sucrose stearate | 9.1 | g |
| Poloxamer 188 | 5.3 | g |
| Kaolin | 35.4 | g |
| Magnesium oxide | 36.2 | g |
| Microcrystalline cellulose | 59.1 | g |
| Lactose | 61.4 | g |
| Starch | 29.5 | g |
| Polyvinyl pyrrolidone | 10.2 | g |

The dispersing capacity of a number of tablets and the suspending capability of the dispersions prepared from the tablets were determined according to the methods described in Example 1. The results show that all tested tablets were disintegrated in water within 13 seconds to form evenly suspending dispersions each having a sedimentation ratio of 0.95.

### EXAMPLE 8

Irisquinone, soy bean lecithin, Tween 60, silicon dioxide, and calcium hydrogen phosphate (amounts indicated in Table 8) were mixed under high shearing, refrigerated at −4° C., smashed, and sieved through 100 meshes. The mixture was then dry-blended with microcrystalline cellulose, sucrose, low-substituted hydroxypropyl cellulose (amounts also indicated in Table 8), granulated, and loaded into gelatin capsules.

TABLE 8
Formulation containing two surfactants
(soy bean lecithin, HLB = 3.5; Tween 60, HLB = 14.9)

| | | |
|---|---|---|
| Irisquinone | 10.0 | g |
| Soy bean lecithin | 0.9 | g |
| Tween 60 | 0.3 | g |
| Silicon dioxide | 40.0 | g |
| Calcium hydrogen phosphate | 60.0 | g |
| Microcrystalline cellulose | 109.8 | g |
| Sucrose | 70.0 | g |
| Low-substituted hydroxypropyl cellulose | 9.0 | g |

The dispersing capacity of a number of capsules and the suspending capability of the dispersions prepared from the capsules were determined according to the methods described in Example 1. The results show that all tested capsules were disintegrated in water within 16 seconds to form evenly suspending dispersions each having a sedimentation ratio of 0.96.

### EXAMPLE 9

Irisquinone, sucrose monostearte, poloxamer 188, and magnesium oxide (amounts indicated in Table 9) were mixed under high shearing, refrigerated at −4° C. smashed, and sieved through 100 meshes. The mixture was dry-blended with microcrystalline cellulose, lactose, croscarmellose sodium (amounts also indicated in Table 9), granulated, and sieved through 22 meshes to obtain granules.

TABLE 9
Formulation containing two surfactants (sucrose stearate,
HLB = 3; and poloxamer 188, HLB = 29)

| | |
|---|---|
| Irisquinone | 100.2 g |
| Sucrose stearate | 20.8 g |
| Poloxamer 188 | 7.7 g |
| Magnesium oxide | 98.6 g |
| Microcrystalline cellulose | 121.3 g |
| Lactose | 39.4 g |
| Croscarmellose sodium | 12.0 g |

The dispersing capacity of the granules and the suspending capability of each dispersion prepared from the granules were determined in a similar manner described in Example 1, except that 0.4 g of the granules, instead of a tablet, were placed in 50 ml of water. The results show that the tested granules were disintegrated in water within 15 seconds to form evenly suspending dispersions each having a sedimentation ratio of 0.97.

### EXAMPLE 10

Granules were prepared from the materials listed in Table 10 according to the method described in Example 9.

TABLE 10
Formulation containing two surfactants (Span 80,
HLB = 4.3; Tween 80, HLB = 15)

| | |
|---|---|
| Irisquinone | 300.0 g |
| Span 80 | 36.7 g |
| Tween 80 | 12.0 g |
| Silicon dioxide | 96.5 g |
| Magnesium oxide | 96.0 g |
| Microcrystalline cellulose | 480.0 g |
| Lactose | 107.5 g |
| Croscarmellose sodium | 71.3 g |

The dispersing capacity of the granules and the suspending capability of the dispersion prepared from the granules were determined according to the methods described in Example 9. The results show that the tested granules were disintegrated in water within 12 seconds to form evenly suspending dispersions each having a sedimentation ratio of 0.93.

### EXAMPLE 11

Irisquinone, sucrose monopalmitate, Span 60, silicon dioxide, and magnesium oxide (amounts indicated in Table 11) were mixed under high shearing, refrigerated at −4° C., smashed, and sieved through 100 meshes. The mixture was then dry-blended with powder cellulose, lactose, and alginate sodium (amounts also indicated in Table 11) to obtain granules.

TABLE 11
Formulation containing two surfactants (Span 60,
HLB = 4.7; sucrose monopalmitate, HLB = 15)

| | |
|---|---|
| Irisquinone | 500.0 g |
| Span 60 | 61.2 g |
| Sucrose monopalmitate | 89.6 g |
| Silicon dioxide | 125.1 g |
| Magnesium oxide | 123.2 g |
| Powder cellulose | 501.4 g |
| Lactose | 499.5 g |
| Alginate sodium | 100.0 g |

The dispersing capacity of the granules and the suspending capability of the dispersion prepared from the granules were determined according to the methods described in Example 10. The results show that the tested granules were disintegrated in water within 14 seconds to form evenly suspending dispersions each having a sedimentation ratio of 0.94.

### EXAMPLE 12

1000 tablets were prepared using the materials listed in Table 12 according to the method described in Example 1.

TABLE 12
Formulation containing two surfactants (sucrose stearate,
HLB = 3; poloxamer 188, HLB = 29)

| | |
|---|---|
| Irisquinone | 40.2 g |
| Sucrose stearate | 9.1 g |
| Poloxamer 188 | 5.3 g |
| Magnesium oxide | 100.5 g |
| Microcrystalline cellulose | 49.2 g |
| Lactose | 51.7 g |
| Starch | 33.8 g |
| Polyvinyl pyrrolidone | 10.2 g |

The dispersing capacity of a number of tablets and the suspending capability of the dispersions prepared from the tablets were determined according to the methods described in Example 1. The results show that all tested tablets were disintegrated in water within 13 seconds to form evenly suspending dispersions each having a sedimentation ratio of 0.95.

### EXAMPLE 13

1000 tablets were prepared using the materials listed in Table 13 according to the method described in Example 1.

TABLE 13
Formulation containing two surfactants (lecithin,
HLB = 3.5; poloxamer 188, HLB = 29)

| | |
|---|---|
| Irisquinone | 32.8 g |
| Lecithin | 3.0 g |
| Poloxamer 188 | 2.2 g |
| Silicon dioxide | 50.4 g |
| Calcium hydrogen phosphate | 45.0 g |
| Microcrystalline cellulose | 116.5 g |
| Lactose | 41.6 g |
| Sodium starch glycolate | 8.5 g |

The dispersing capacity of a number of tablets and the suspending capability of the dispersions prepared from the tablets were determined according to the methods described in Example 1. The results show that all tested tablets were disintegrated in water within 13 seconds to form evenly suspending dispersions each having a sedimentation ratio of 0.97.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A composition comprising a water-insoluble bioactive agent and two surfactants having different HLB values, wherein the difference between the HLB values of the two surfactants is greater than 5.

2. The composition of claim 1, wherein the bioactive agent is a solid.

3. The composition of claim 1, wherein the bioactive agent is a liquid.

4. The composition of claim 3, further comprising an absorbent.

5. The composition of claim 1, wherein one of the surfactants is a hydrophilic surfactant having an HLB greater than 10, and the other is a lipophilic surfactant having an HLB smaller than or equal to 10.

6. The composition of claim 1, wherein the bioactive agent is irisquinone.

7. The composition of claim 6, further comprising an absorbent.

8. The composition of claim 7, wherein the amount of the surfactants is 0.01 to 0.3 parts per part irisquinone by weight, and the amount of the absorbent is 0.5 to 10.0 parts per part irisquinone by weight.

9. The composition of claim 8, wherein one of the surfactants is a hydrophilic surfactant having an HLB value greater than 10, and the other is a lipophilic surfactant having an HLB value smaller than or equal to 10.

10. The composition of claim 9, wherein the hydrophilic surfactant is Tween 80, sucrose monostearate, or sodium lauryl sulfate; and the lipophilic surfactant is sucrose stearate, lecithin, Span 80, or glycerol monostearate.

11. The composition of claim 10, wherein the hydrophilic and lipophilic surfactants are Tween 80 and sucrose stearate, sucrose monostearate and lecithin, sodium lauryl sulfate and sucrose stearate, Tween 80 and Span 80, Tween 80 and lecithin, sucrose monostearate and glycerol monostearate, or Tween 80 and glycerol monostearate.

12. The composition of claim 11, wherein the absorbent is magnesium oxide, magnesium carbonate, silicon dioxide, magnesium aluminum silicate, calcium carbonate, calcium phosphate, calcium hydrogen phosphate, calcium oxide, calcium hydrogen carbonate, aluminum hydroxide, magnesium hydroxide, Kaolin, or a mixture thereof.

13. The composition of claim 12, wherein the irisquinone is irisquinone A, irisquinone B, or a mixture thereof.

14. The composition of claim 13, wherein the irisquinone is a mixture of irisquinone A and irisquinone B, irisquinone A being 80% to 95% of the mixture by weight.

15. The composition of claim 5, wherein the difference between their HLB values is greater 10.

16. The composition of claim 1, wherein the composition is in form of a tablet.

17. The composition of claim 16, further comprising an additive, wherein the additive is a disintegrator, a diluent, or a dispensing agent.

18. The composition of claim 8, wherein the composition is in form of a tablet.

19. The composition of claim 18, further comprising an additive, wherein the additive is a disintegrator, a diluent, or a dispensing agent.

20. The composition of claim 13, wherein the composition is in the form of a tablet or capsule.

21. The composition of claim 20, further comprising an additive, wherein the additive is a disintegrator, a diluent, or a dispensing agent.

22. A method for preparing a composition of a water-insoluble bioactive agent, comprising:
selecting two surfactants based on the difference between their HLB values being greater than 5; and
blending the two surfactants and a water insoluble bioactive agent to obtain a composition.

23. The method of claim 22, wherein the bioactive agent is a solid.

24. The method of claim 22, wherein the bioactive agent is a liquid.

25. The method of claim 22, wherein an absorbent is blended together with the two surfactants and the bioactive agent.

26. The method of claim 22, wherein the bioactive agent is irisquinone.

27. The method of claim 26, wherein an absorbent is blended together with the two surfactants and the irisquinone.

28. The method of claim 27, wherein the amount of the surfactants is 0.01 to 0.3 parts per part irisquinone by weight, and the amount of the absorbent is 0.5 to 10.0 parts per part irisquinone by weight.

29. The method of claim 28, wherein one of the surfactants is a hydrophilic surfactant having an HLB greater than 10, and the other is a lipophilic surfactant having an HLB smaller than or equal to 10.

30. The method of claim 29, wherein the hydrophilic surfactant is Tween 80, sucrose monostearate, or sodium lauryl sulfate; and the lipophilic surfactant is sucrose stearate, lecithin, Span 80, or glycerol monostearate.

31. The method of claim 30, wherein the hydrophilic and lipophilic surfactants are Tween 80 and sucrose stearate, sucrose monostearate and lecithin, sodium lauryl sulfate and sucrose stearate, Tween 80 and Span 80, Tween 80 and lecithin, sucrose monostearate and glycerol monostearate, or Tween 80 and glycerol monostearate.

32. The method of claim 30, wherein the absorbent is magnesium oxide, magnesium carbonate, silicon dioxide, magnesium aluminum silicate, calcium carbonate, calcium phosphate, calcium hydrogen phosphate, calcium oxide, calcium hydrogen carbonate, aluminum hydroxide, magnesium hydroxide, Kaolin, or a mixture thereof.

33. The method of claim 32, wherein the irisquinone is a mixture of irisquinone A and irisquinone B, irisquinone A being 80% to 95% of the mixture by weight.

34. The method of claim 29, wherein the difference between the HLB values of the two surfactants is greater than 10.

35. The method of claim 22, wherein the blending step is performed under high-speed shearing.

36. The method of claim 35, further comprising freeze-smashing the composition.

37. The method of claim 22, fuirther comprising: compressing the composition to form a tablet after the blending step.

38. The method of claim 37, an additive is compressed together with the composition, wherein the additive is a disintegrator, a diluent, or a dispensing agent.
